(19) United States
(12) Patent Application Publication
(10) Pub. No.: US 2014/0107772 A1
(43) Pub. Date: Apr. 17, 2014

(54) ROUGHENED CUFF SURFACE

(71) Applicant: ST. JUDE MEDICAL, CARDIOLOGY DIVISION, INC., St. Paul, MN (US)

(72) Inventors: XueMei Li, Shoreview, MN (US); Yousef F. Alkhatib, Edina, MN (US); Zhengrong Zhou, Shanghai (CN)

(73) Assignee: St. Jude Medical, Cardiology Division, Inc., St. Paul, MN (US)

(21) Appl. No.: 13/786,999

(22) Filed: Mar. 6, 2013

Related U.S. Application Data
(60) Provisional application No. 61/713,224, filed on Oct. 12, 2012.

(51) Int. Cl. A61F 2/24

(52) U.S. Cl. CPC A61F 2/2418; A61F 2/2415
USPC 623/2.17; 29/428

(57) ABSTRACT

A prosthetic heart valve includes a collapsible and expandable stent having a proximal end, a distal end, an annulus section adjacent the proximal end and an aortic section adjacent the distal end. A cuff having an inner surface and an outer surface is made sufficiently rough to promote tissue growth. The prosthetic heart valve further includes a collapsible and expandable valve assembly, the valve assembly including a plurality of leaflets connected to at least one of the stent and the cuff.

### CROSS-REFERENCE TO RELATED APPLICATION

The application claims the benefit of the filing date of U.S. Provisional Patent Application No. 61/713,224 filed Oct. 12, 2012, the disclosure of which is hereby incorporated herein by reference.

### BACKGROUND OF THE INVENTION

The present invention relates to heart valve replacement and, in particular, to collapsible prosthetic heart valves. More particularly, the present invention relates to collapsible prosthetic heart valves with superior sealing.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two common types of stents on which the valve structures are ordinarily mounted: a self-expanding stent and a balloon-expandable stent. To place such valves into a delivery apparatus and ultimately into a patient, the valve must first be collapsed or crimped to reduce its circumferential size.

When a collapsed prosthetic valve has reached the desired implant site in the patient (e.g., at or near the annulus of the patient's heart valve that is to be replaced by the prosthetic valve), the prosthetic valve can be deployed or released from the delivery apparatus and re-expanded to full operating size. For balloon-expandable valves, this generally involves releasing the valve, assuring its proper location, and then expanding a balloon positioned within the valve stent. For self-expanding valves, on the other hand, the stent automatically expands as the sheath covering the valve is withdrawn. Examples of collapsible heart valves can be found in, for example, U.S. Pat. Nos. 5,411,552, 7,892,281, 8,002,825, 7,393,360, 7,914,575, 6,458,153 6,267,253, U.S. Patent Publication No. 2011/0022157 and U.S. patent application Ser. No. 11/128,826.

Despite the various improvements that have been made to the collapsible prosthetic heart valve, common devices suffer from some shortcomings. For example, in some conventional prosthetic valves a polymeric cuff is attached to the stent. After implantation, small gaps formed between the cuff and the site of implant may cause complications such as paravalvular leakage, blood flowing through a channel between the structure of the implanted valve and cardiac tissue as a result of a lack of appropriate sealing. This leakage can have severely adverse clinical outcomes. To reduce these adverse events, a valve should seal and adequately anchor within the annulus without the need for excessive radial forces that could harm nearby anatomy or physiology.

There therefore is a need for further improvements to collapsible prosthetic heart valves, and in particular, to cuffs of prosthetic heart valves. Among other advantages, the present invention may address one or more of these needs.

### SUMMARY OF THE INVENTION

The invention includes a cuff useful in a prosthetic heart valve which has been roughened or textured beyond any natural topography that may exist. By altering the surface topography, cell growth between the heart valve and the surrounding anatomy is promoted to assist in effectively sealing the area between the prosthetic valve and tissue. Superior sealing due to tissue growth between the valve and the anatomy allows the valve prosthetic heart valve to function as intended without the risk of paravalvular leakage for a longer period of time. Various methods and techniques are disclosed for roughening or texturing the cuff.

In some embodiments, a prosthetic heart valve includes a collapsible and expandable stent having a proximal end, a distal end, an annulus section adjacent the proximal end and an aortic section adjacent the distal end. The heart valve further includes a cuff having an inner surface and an outer surface, the outer surface having indentations capable of providing a rough surface to promote tissue growth and a collapsible and expandable valve assembly, the valve assembly including a plurality of leaflets connected to at least one of the stent and the cuff. The cuff maybe attached to the luminal or ablumental surface of the valve.

By “indentation” it will be understood that any form of artificially roughened surface is contemplated such that the topography of the outer surface is no longer the same as the inner surface and is different than prior to roughening. In some examples, the indentations are uniformly distributed on the cuff. Some or substantially all of the outer surface may be roughened with indentations and the amount of roughening may vary on a single cuff. The indentations may be of any depth relative to the outer surface of the cuff and may extend partially or fully through the thickness of the cuff. The outer surface of the cuff may be rough at a microscopic or macroscopic level. In some examples, the cuff is formed of a polymer such as a polyurethane or a silicone.

In some embodiments, a method of treating a cuff to provide indentations includes providing a collapsible and expandable stent having a proximal end, a distal end, an annulus section adjacent the proximal end and an aortic section adjacent the distal end, roughening an outer surface of a cuff to promote tissue growth between the prosthetic valve and the tissue and coupling the cuff to the collapsible and expandable stent. The cuff can also be roughened once coupled to the strut.

Roughening an outer surface of a cuff may include forming indentations in the cuff at a macroscopic or microscopic level. Roughening an outer surface of a cuff may include using at least one needle to puncture the cuff or a thermal treatment to alter the outer surface of the cuff. A surface coating technique may also alter the outer surface of the cuff. A chemical vapor deposition technique may be used to roughen the cuff. Any other techniques capable of producing indentations are contemplated.

In at least some examples, a gas may be used to treat the surface and generate bioactive groups or chemical structures on the cuff. The roughening step may also include immobilization of biological molecules such as growth factors onto the cuff to promote tissue growth. The roughening step may also include releasing biological molecules at a device-tissue interface to promote tissue growth.

### BRIEF DESCRIPTION OF THE DRAWINGS

Various embodiments of the presently disclosed delivery system are disclosed herein with reference to the drawings, wherein:

FIG. 1 is a partial side elevational view of a prosthetic heart valve including a stent and a valve assembly having a cuff and leaflets;

FIG. 2A is a perspective side view of a cuff prior to attachment to a heart valve;

FIG. 2B is a perspective side elevational view of a cuff after the attachment portions of the cuff have been coupled together;

FIG. 3 is a perspective side view of a cuff coupled to a stent via sutures;

FIG. 4 is a perspective side view of a cuff coupled to a stent via sutures, the cuff having trimmed portions;

FIG. 5 is a perspective side view of a portion of a prior art prosthetic heart valve, showing gaps formed between the valve and surrounding tissue;

FIG. 6A is a cross-sectional view of a cuff prior to indenting and needles for indenting the cuff;

FIG. 6B is a cross-sectional view of the cuff of FIG. 6A after the cuff has been indented using the needles;

FIG. 6C is perspective side view of a cuff having indentations; and

FIG. 7 is a perspective side view of a portion of a prosthetic heart valve having a cuff that has promoted tissue growth to seal the valve.

Various embodiments of the present invention will now be described with reference to the appended drawings. It is appreciated that these drawings depict only some embodiments of the invention and are therefore not to be considered limiting of its scope.

### DETAILED DESCRIPTION OF THE INVENTION

As used herein, the term “proximal,” when used in connection with a prosthetic heart valve, refers to the end of the heart valve closest to the heart when the heart valve is implanted in a patient, whereas the term “distal,” when used in connection with a prosthetic heart valve, refers to the end of the heart valve farthest from the heart when the heart valve is implanted in a patient.

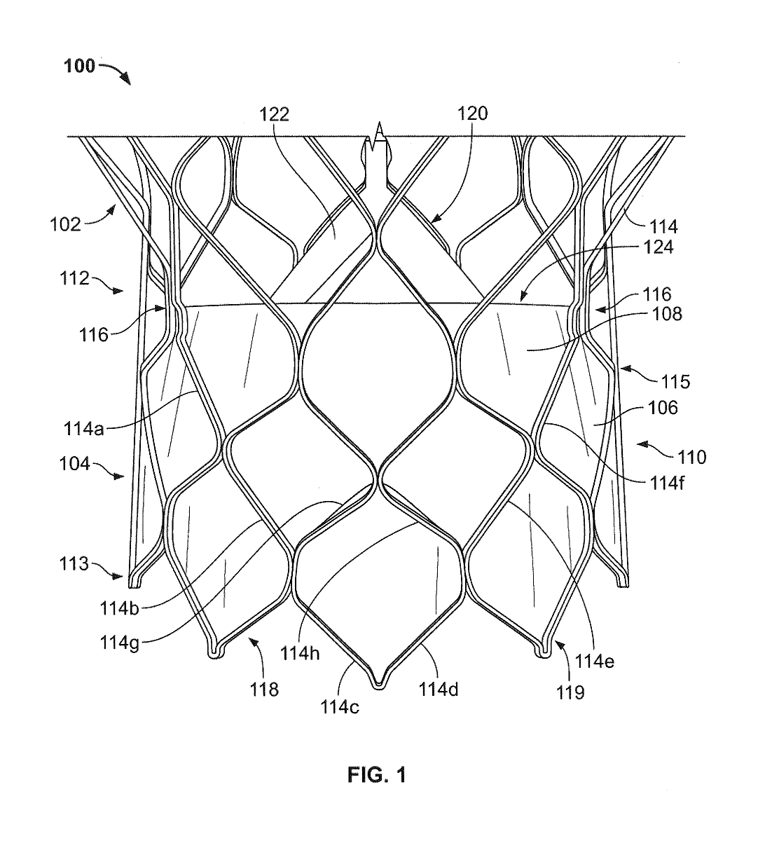

FIG. 1 shows a collapsible prosthetic heart valve 100 according to an embodiment of the present disclosure. The prosthetic heart valve 100 is designed to replace the function of a native aortic valve of a patient. Examples of collapsible prosthetic heart valves are described in International Patent Application Publication No. WO/2009/042196; U.S. Pat. No. 7,018,406; and U.S. Pat. No. 7,329,278, the disclosures of all of which are hereby incorporated herein by reference. As discussed in detail below, the prosthetic heart valve has an expanded condition and a collapsed condition. Although the invention is described herein as applied to a prosthetic heart valve for replacing a native aortic valve, the invention is not so limited, and may be applied to prosthetic valves for replacing other types of implantable valves, cardiac and otherwise.

The prosthetic heart valve 100 includes a stent or frame 102, which may be wholly or partly formed of any biocompatible material, such as metals, synthetic polymers, or biopolymers capable of functioning as a stent. Suitable biopolymers include, but are not limited to, elastin, and mixtures or composites thereof. Suitable metals include, but are not limited to, cobalt, titanium, nickel, chromium, stainless steel, and alloys thereof, including nitinol. Suitable synthetic polymers for use as a stent include, but are not limited to, thermoplastics, such as polyolefins, polyesters, polyamides, polysulfones, acrylics, polyacrylonitriles, polyetheretherketone (PEEK), and polyaramides. The stent 102 may have an annulus section 110, an aortic section (not shown) and a transition section (not shown) disposed between the annulus section and the aortic section. Each of the annulus section 110, the aortic section and the transition section of the stent 102 includes a plurality of cells 112 connected to one another around the stent. The annulus section 110 and the aortic section of the stent 102 may include one or more annular rows of cells 112 connected to one another. For instance, the annulus section 110 may have two annular rows of cells 112. When the prosthetic heart valve 100 is in the expanded condition, each cell 112 may be substantially diamond shaped. Regardless of its shape, each cell 112 is formed by a plurality of struts 114. For example, a cell 112 may be formed by four struts 114.

The stent 102 may include commissure features 116 connecting at least two cells 112 in the longitudinal direction of the stent 102. The commissure features 116 may include eyelets for facilitating the suturing of a valve assembly 104 to the sent 102.

The prosthetic heart valve 100 also includes a valve assembly 104 attached inside the annulus section 110 of the stent 102. United States Patent Application Publication No. 2008/0228264, filed Mar. 12, 2007, and United States Patent Application Publication No. 2008/0147179, filed Dec. 19, 2007, the entire disclosures of both of which are hereby incorporated herein by reference, describe suitable valve assemblies. The valve assemblies can also be bound to the stent through chemical bounds using dip-coating process. The valve assembly 104 may be wholly or partly formed of any suitable biological material or polymer. Examples of biological materials suitable for the valve assembly 104 include, but are not limited to, porcine or bovine pericardial tissue. Examples of polymers suitable for the valve assembly 104 include, but are not limited to, polyurethane, silicone, and polyester. In at least some examples, portions of valve assembly 104, a cuff and the suture used may include an ultra high molecular weight polyethylene, such as FORCE FIBER®.

The valve assembly 104 may include a cuff 106 disposed on the lumenal surface of annulus section 110, on the ablumenal surface of annulus section 110, or on both surfaces, and the cuff may cover all or part of either or both of the lumenal and ablumenal surfaces of the annulus section. The cuff 106 and/or the sutures used to attach the valve assembly 104 to stent 102 may be formed from polyurethane copolymers or include ultra high molecular weight polyethylene as well as any of the materials discussed above with reference to valve assembly 104. FIG. 1 shows cuff 106 disposed on the lumenal surface of annulus section 110 so as to cover part of the annulus section while leaving another part thereof uncovered. The cuff 106 may be attached to strut 102 by dip-coating the polymer onto the stent or by one or more strings or sutures passing through the cuff and around selected struts 114 of the stent. The valve assembly 104 may further include a plurality of leaflets 108 which collectively function as a one-way valve. A first edge 122 of each leaflet 108 may be attached to the stent 102 between two adjacent commissure features 116 by any suitable attachment means, such as suturing, stapling, adhesives or the like. For example, the first edge 122 of each leaflet 108 may be bound by dip-coating through a leaflet shaped mandrel. In another example, the first edge 122 of each leaflet 108 may be sutured to the stent 102 by passing strings or sutures through the cuff 106 of the valve assembly 104. The leaflets 108 may be attached to the stent 102 along at least some struts 114 of the stent and through the eyelets in the commissure features 116 to enhance the structural integrity of the valve assembly 104. A second or free edge 124 of each leaflet 108 may coapt with the corresponding free edges of the other leaflets, thereby enabling the leaflets to function collectively as a one-way valve.

As shown in FIG. 1, at least one leaflet 108 may be attached to the stent 102 so that its first edge 122 is disposed substantially along specific struts 114a, 114b, 114c, 114d, 114e and 114f located in the annulus section 110 of the stent. That is, the edge 122 is positioned in substantial alignment with struts 114a, 114b, 114c, 114d, 114e, and 114f. Struts 114a, 114b, and 114c may be connected to one another in substantially end-to-end fashion diagonally along three cells 112, beginning with an end of the strut 114a connected to a commissure feature 116 and ending with an end of strut 114c connected to an end of strut 114d. Struts 114c and 114d are part of the same cell 112 and may collectively define a substantially right angle between them. Struts 114d, 114e, and 114f may be connected to one another in substantially end-to-end fashion diagonally along three cells 112, beginning with an end of the strut 114f connected to a commissure feature 116 and ending with the connection between an end of strut 114c and an end of strut 114d.

As discussed above, the leaflets 108 may be attached directly to and supported by the struts 114a, 114b, 114c, 114d, 114e, and 114f, and by commissure features 116, such as by suturing. In such event, the cuff 106 may perform little or no supportive function for the leaflets 108. Hence, the cuff 106 is not subjected to high stresses and is therefore less likely to fail during use. In light of this, the thickness of the cuff may be reduced. Reducing the thickness of the cuff 106 results in a decrease in the volume of the valve assembly 104 in the collapsed condition. This decreased volume is desirable as it enables the prosthetic heart valve 100 to be implanted in a patient using a delivery device that is smaller in cross-section than conventional delivery devices. In addition, since the material forming the stent struts 114 is stronger than the material forming the cuff 106, the stent struts 114 may perform the supportive function for the leaflets 108 better than the cuff 106.

In operation, the embodiments of the prosthetic heart valve 100 described above may be used to replace a native heart valve, such as the aortic valve, a surgical heart valve or a heart valve that has undergone a surgical procedure. The prosthetic heart valve may be delivered to the desired site (e.g., near a native aortic annulus) using any suitable delivery device. During delivery, the prosthetic heart valve is disposed inside the delivery device in the collapsed condition. The delivery device may be introduced into a patient using a transfemoral, transapical, transseptal or other approach. Once the delivery device has reached the target site, the user may deploy the prosthetic heart valve. Upon deployment, the prosthetic heart valve expands into secure engagement within the native aortic annulus. When the prosthetic heart valve is properly positioned inside the heart, it works as a one-way valve, allowing blood to flow in one direction and preventing blood from flowing in the opposite direction.

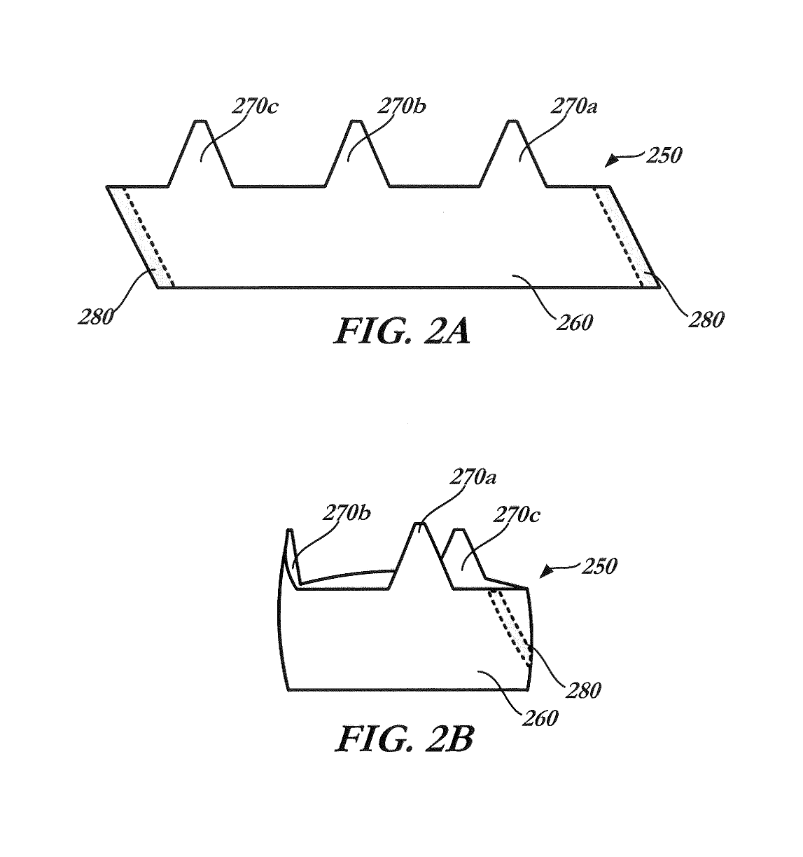

FIG. 2A illustrates the outer diameter of a cuff 250 before coupling to a stent (not shown). In this example, cuff 250 includes an elongated body 260 in the shape of a parallelogram though it will be understood that body 260 may be formed in any other suitable shape such as other quadrilaterals, a triangle or an oval. Cuff 250 may also include a series of triangular-shaped posts 270a, 270b, 270c for coupling cuff 250 to commissure features (not shown). Again, it will be understood that the shape of posts 270 may be varied as desired and that other shapes such as ovals, squares or rectangles may be used to form posts 270. Cuff 250 further includes a pair of attachment portions 280 formed as strips on opposite sides of body 260.

As seen in FIG. 2A, cuff 250 is configured to include two complementary attachment portions 280 such that the cuff 250 may form a wrapped configuration when the attachment portions 280 are coupled together. FIG. 2B, shows the cuff 250 of FIG. 2A in this wrapped configuration. Attachment portions 280 may be coupled together using a suture, a staple, an adhesive or any other suitable means. In at least some other examples, the attachment portions 280 are coupled to each other and to selected struts of a stent.

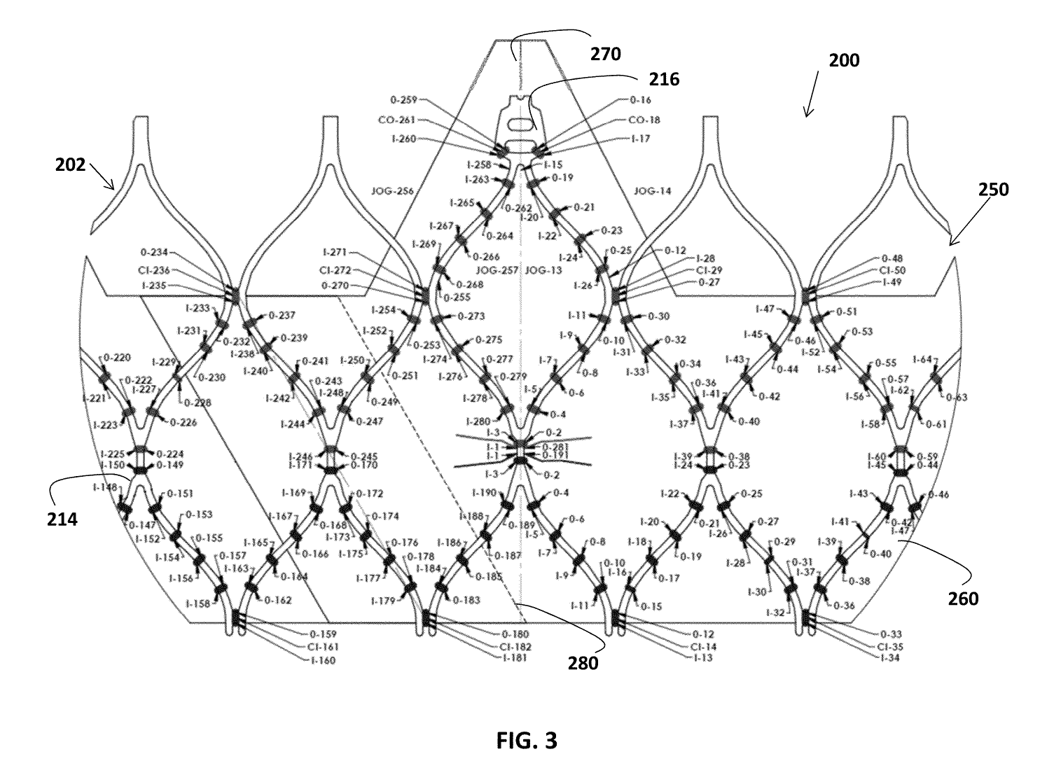

As seen in FIG. 3, cuff 250 may be coupled to portions of stent 202 using sutures. In some examples, body 260 of cuff 250 may be coupled to the stent 202 using sutures along struts 214 of stent 202. Cuff 250 may also be coupled to commissure features 216 of stent 202 along posts 270. While FIG. 3 illustrates the cuff 250 being disposed on the lumenal surface of stent 202, it will be understood that cuff 250 may instead be disposed on the ablumenal surface of stent 202. Additionally, it is contemplated that two cuffs may be disposed on stent 202, one on each of the lumenal and ablumenal surfaces.

In at least some examples, attachment portions 280 are coupled together using the above-described techniques prior to suturing cuff 250 to stent 202. Alternatively, attachment portions 280 may be coupled together after cuff 250 has been sutured to stent 202.

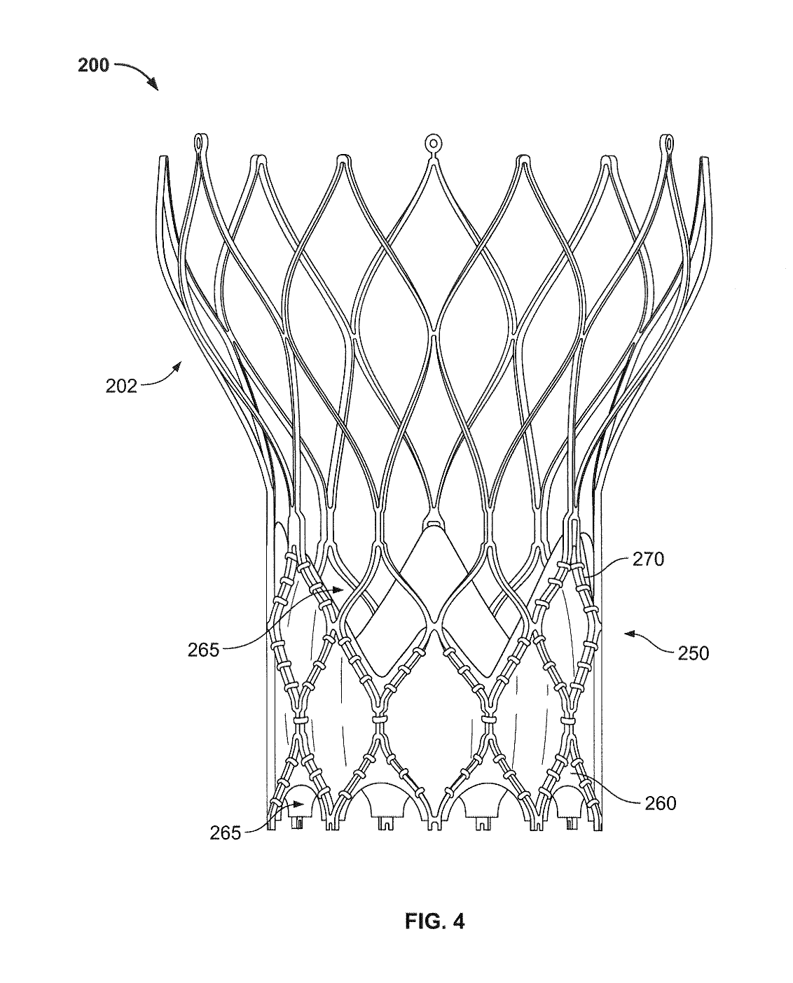

Prior or after attachment of cuff 250 to stent 202, portions of body 260 of the cuff 250 may be trimmed. Using a cutting mandrel and/or die, portions of body 260 corresponding to the certain cells of the prosthetic heart valve 200 may be trimmed. FIG. 4 illustrates a prosthetic heart valve 200 including a stent 202 and a cuff 250, the cuff 250 having trimmed portions 265 near the proximal end. As seen in FIG. 4, trimmed portions 265 may be formed as semicircular cutouts at the bottom of cuff 250, corresponding to the most-proximal cells of stent 202. Alternatively, trimmed portions 265 may include triangular cutouts. Trimmed portions 265 may also form a shape that follows the struts of 202 so as to remove as much of the unused cuff as possible to reduce bulk. A comparison of FIGS. 3 and 4 illustrates that distal portions of cuff 250 may also include trimmed portions 265 at certain cells. With cuff 250 attached to stent 202, leaflets (not shown) may be attached to the cuff to complete assembly of the heart valve. The foregoing, however, is for illustrative purposes only and it will be understood that the present invention may be useful for various constructions of a prosthetic heart valve 200.

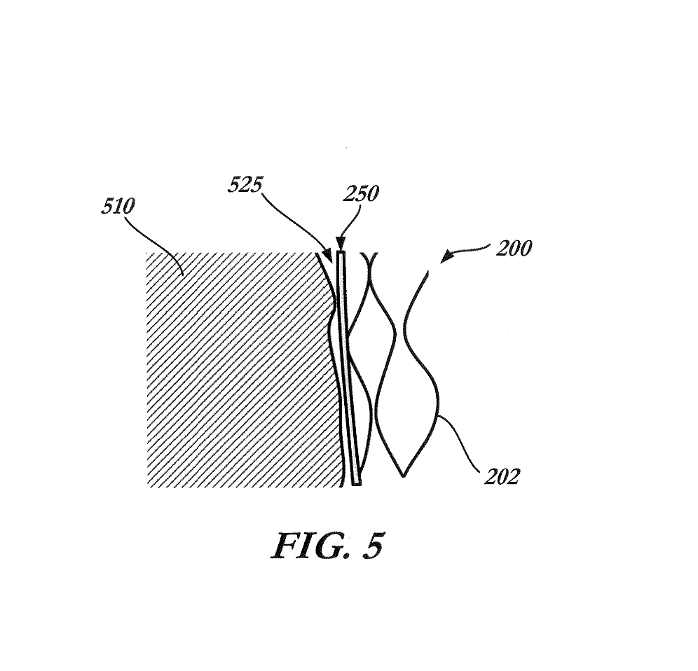

FIG. 5 illustrates a portion of a conventional prosthetic heart valve 200 having a stent 202, a cuff 250 and leaflets (not shown) disposed within patient anatomy near tissue 510. For the sake of clarity, only a portion of cuff 250 is shown, although it will be understood that cuff 250 wraps around the perimeter of heart valve 200.

As seen in FIG. 5, the use of conventional prosthetic heart valves having polymeric cuffs may result in small gaps 525 disposed between the heart valve 200 and tissue of the annulus or trapped valve leaflets 510. Specifically, gaps 525 due to the manufacturing methods of conventional cuffs may be formed near the aortic root and the implanted valve 200 even in cases where valve fitment and placement are satisfactory. For example, in manufacturing the prosthetic heart valve the polymeric cuff and/or leaflets may be dip coated separately or together to produce thin films. After the polymers cure, a smooth thin film is formed, which plays a sealing role after implantation. However, dip coated smooth cuffs may inhibit tissue growth and compromise long-term sealing of percutaneous polymer valve, leaving gaps 525 between the tissue and the valve 200 as shown in FIG. 5. These gaps 525 may in turn cause paravalvular leakage. Though fabric cuffs may promote tissue growth, they are typically thicker and less flexible, and thus add bulk to the heart valve assembly and thereby increase the crimping profile of the stent. Some cuffs may be made from sheets of polymers that need not be dipped or from a woven material. Additionally, the cuff may not necessarily be trimmed.

Examples of methods for promoting tissue growth in cuffs to improve valve sealing while minimizing the crimping profile include the following. In some examples, promoting tissue growth is accomplished by indenting the cuff at microscopic or molecular levels using physical, chemical or biological means.

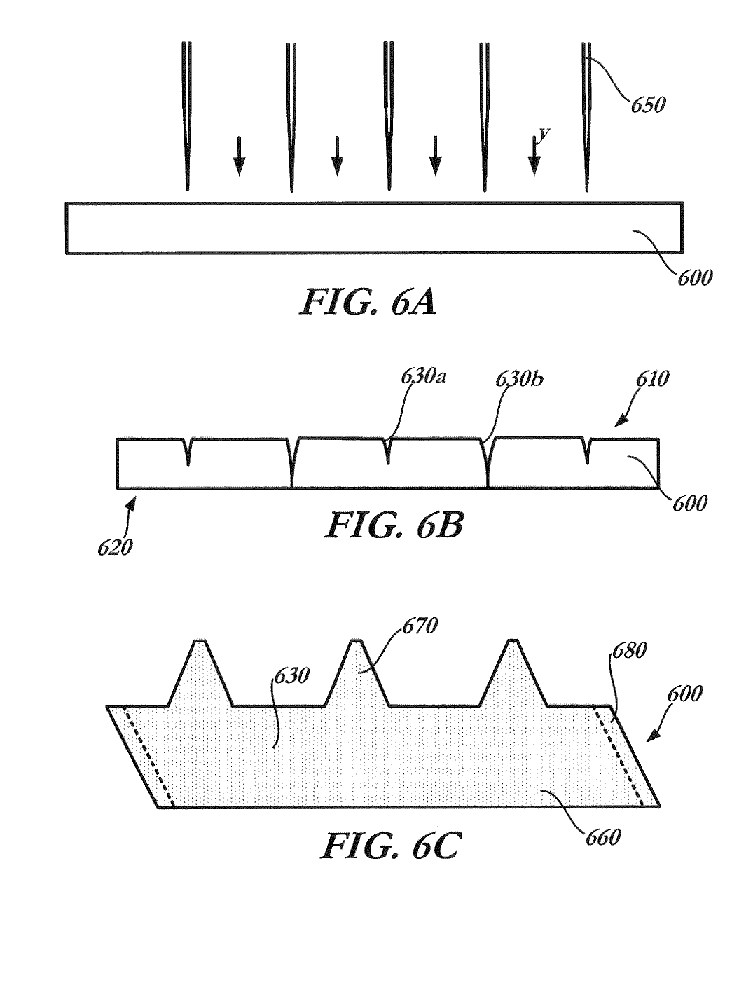

In a first example, cell growth between a prosthetic heart valve and the patient anatomy may be promoted by using mechanical means to form indentations on a surface of a cuff. FIG. 6A illustrates a cross-sectional view of a cuff 600 prior to indentation. Though the following examples describe polymeric cuffs, it will be understood that the principles discussed herein may be equally applicable to polymer cuff as well as biological cuffs (e.g., porcine and bovine cuffs). In at least some examples, the cuff and the leaflets are formed of the same material. Any polymer having sufficient thin film strength that will not be damaged during crimping and deploying may be selected. In at least some examples, the material for the cuff and/or leaflet may be selected from polyurethanes (e.g., Elast-Eon), silicones, fluoro-polymers, polyesters (e.g., PET) or thermoplastic polymers, such as polyethylenes.

As seen in FIG. 6A, a plurality of needles 650 may be used to indent or perforate the cuff 600 at various points to form a roughened or textured cuff and promote tissue growth. To roughen the cuff 600, needles 650 may pierce cuff 600 by traveling in direction y at least partially through cuff 600. As seen in FIG. 6B, indentations 630 are formed in cuff 600 as a results of the piercing of the needles 650. Indentations 630 may be formed at the surface of cuff 600 as illustrated by indentations 630a, or extend completely through from the top 610 of the cuff 600 to the underside 620 of cuff 600 to form holes or perforations as illustrated by indentations 630b. It will be understood that the indentations 630 may be formed in any part and on any portion of cuff 600 where tissue growth is to be promoted. In at least some examples, the ablumenal side of the cuff is roughened or indented. Alternatively, both the ablumenal and lumenal sides of the cuff are roughened.

FIG. 6C illustrates a cuff 600 having indentations 630 uniformly disposed on the ablumenal surface of the cuff 600. As seen in FIG. 6C, indentations 630 may be formed on any of body 660, posts 670 and/or attachment portions 680. It will be understood that instead of uniformly distributed indentations 630, such indentations may be randomly formed in cuff 600. Additionally, the density and location of the indentations may be varied as desirable. In addition to the needle indentation described above, other mechanical means such as dipping, mandrel surface patterning or surface etching may also be used to roughen the cuff at the macroscopic level. Sandpaper may also be used to roughen the cuff.

Cell growth between a prosthetic heart valve and the patient anatomy may also be promoted by using other physical, chemical and/or biological methods at a microscopic or molecular level (e.g., sub-micrometer or nanometer scale). Such indentations not only allow for a reduction in valve profile, but provide greater flexibility in choosing a material to form the cuff and effectively promote tissue growth.

In at least some examples, physical means may be used to indent or roughen the cuff. Specifically, selectively altering surface morphology or topology, molecular orientation, alignment or surface chemistry may create a desired surface pattern and/or molecular structure that may potentially enhance tissue growth at the device-tissue interface. Suitable physical methods for modifying the cuff may include, but are not limited to the use of thermal treatment, laser surface treatment, and/or exposure to UV light or other radiation. As described above, such treatment may be applied to any portion of the cuff including any of the body, the posts or the attachment portions.

The desired surface modification may also be accomplished using chemical means. For example, an acid etch may be used to form perforations on the cuff. Additionally, in at least some examples, the cuff is modified using surface coating such as chemical vapor deposition. At a gas phase, chemicals may be deposited to the cuff surface, which contain bioactive functional groups or structures (e.g., an amine, amide, ester, carboxylic, urea, urethane, etc.). The surface of the cuff may also be modified using plasma, gas or chemical treatment. For example, using a gas such as oxygen, nitrogen, ammonia, or the like, to treat the surface and to generate bioactive groups or chemical structures. At a solution phase, pendant or comb-like molecular structures may be attached to the cuff surface via surface-initiated polymerization, molecular grafting or surface reaction and immobilization to roughen the surface of the cuff.

The cuff may also be modified using biological means. For example, a chemical or physical treatment as described above may be followed by immobilization of biological molecules, for example, growth factors, onto the cuff surface via covalent or hydrogen bonding, static interactions, molecular interpenetrating networks, or the like. Biological molecules may also be selectively recruited or released at the device-tissue interface to promote tissue growth.

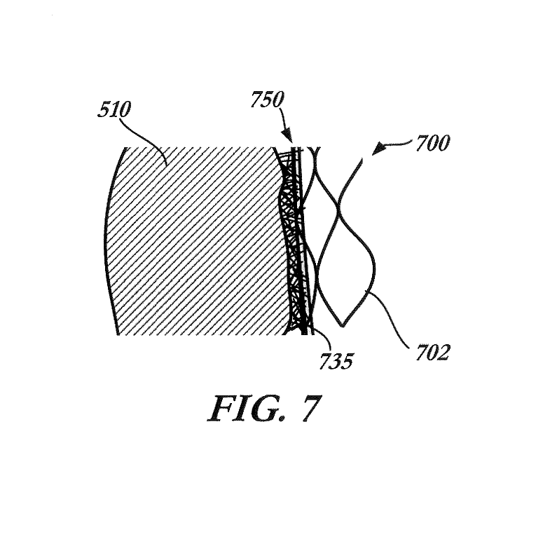

FIG. 7 illustrates a portion of a prosthetic heart valve 700 having a stent 702, a roughened cuff 750 and leaflets (not shown for the sake of clarity) disposed within patient anatomy near tissue 510. As will be appreciated from FIG. 7, the small gaps seen in FIG. 5 are no longer present between the heart valve 700 and tissue 510. Instead, roughened cuff 750 has promoted tissue growth 735 between heart valve 700 and tissue 510, effectively sealing the area between the valve and tissue. Superior sealing due to tissue growth 735 allows the valve prosthetic heart valve 700 to function as intended without the risk of paravalvular leakage for a longer period of time.

Although the invention herein has been described with reference to particular embodiments, it is to be understood that these embodiments are merely illustrative of the principles and applications of the present invention. For example, though the preceding examples have illustrates heart valves, it will be understood that the present invention may be useful in altering cuff surface topography of any other type of valve or other implantable device (e.g., annuloplasty rings) where cellular growth is to be encouraged.

Moreover, any of the treatments discussed above may be applied to any portion of the cuff including any of the body, the posts or the attachment portions. Additionally, a cuff may be subjected to any combination of the treatments illustrated above. For example, a cuff may be subjected to microscopic-level treatment such as laser surface modification as well as macroscopic-level treatment such as needle piercing. Additionally, different portions of the cuff may be subjected to varying methods of treatment. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A prosthetic heart valve, comprising:
a collapsible and expandable stent having a proximal end and a distal end;
a cuff having an inner surface and an outer surface, at least a portion of the outer surface having a plurality of indentations capable of promoting tissue growth connected to at least one of the inner and outer surfaces of the stent;
a collapsible and expandable valve assembly, the valve assembly including a plurality of leaflets connected to at least one of the stent and the cuff.

2. The prosthetic heart valve of claim 1, wherein the plurality of indentations are uniformly distributed on the cuff.

3. The prosthetic heart valve of claim 1, wherein the indentations extend partially through the thickness of the cuff.

4. The prosthetic heart valve of claim 2, wherein the indentations extend fully through the thickness of the cuff.

5. The prosthetic heart valve of claim 1, wherein the outer surface of the cuff is rough at a microscopic level.

6. The prosthetic heart valve of claim 1, wherein the outer surface of the cuff is rough at a macroscopic level.

7. The prosthetic heart valve of claim 1, wherein the cuff is formed of a polymer.

8. The prosthetic heart valve of claim 7, wherein the polymer comprises polyurethane.

9. The prosthetic heart valve of claim 7, wherein the polymer comprises a silicone.

10. A method of treating a cuff for a prosthetic valve assembly comprising:
providing a collapsible and expandable stent having a proximal end and a distal end;
roughening at least a portion of an outer surface of a cuff to promote tissue growth on the roughened surface;
coupling the cuff to the collapsible and expandable stent.

11. The method of claim 10, wherein the cuff is coupled to the stent after the outer surface of the cuff has been roughened.

12. The method of claim 10, wherein roughening an outer surface of a cuff comprises forming indentations in the cuff at a macroscopic level.

13. The method of claim 10, wherein roughening an outer surface of a cuff comprises forming indentations in the cuff at a microscopic level.

14. The method of claim 10, wherein roughening an outer surface of a cuff comprises using at least one needle to puncture the cuff.

15. The method of claim 10, wherein roughening an outer surface of a cuff comprises using a thermal treatment to alter the outer surface of the cuff.

16. The method of claim 10, wherein roughening an outer surface of a cuff comprises using a surface coating technique to alter the outer surface of the cuff.

17. The method of claim 10, wherein roughening an outer surface of a cuff comprises using a chemical vapor deposition technique to roughen the cuff.

18. The method of claim 10, wherein roughening an outer surface of a cuff comprises using a gas to treat the surface and generate bioactive groups or chemical structures on the cuff.

19. The method of claim 10, wherein roughening an outer surface of a cuff comprises immobilization of biological molecules onto the cuff to promote tissue growth.

20. The method of claim 19, wherein immobilization of biological molecules comprises treating the cuff with growth factors.

21. The method of claim 10, wherein roughening an outer surface of a cuff comprises releasing biological molecules at a device-tissue interface to promote tissue growth.
